(19) United States
(12) Patent Application Publication
(10) Pub. No.: US 2016/0213711 A1
(43) Pub. Date: Jul. 28, 2016

(54) Cancer Therapy With Silver Nanoparticles

(71) Applicants: Ilaria E. Palama, Racale (Le) (IT); Mauro Pollini, Merine (Le) (IT); Federica Paladini, Surbo (Le) (IT); Gianluca Accorsi, Nardo (Le) (IT); Alessandro Sannino, Lecce (IT); Giuseppe Gigli, Lecce (IT)

(72) Inventors: Ilaria E. Palama, Racale (Le) (IT); Mauro Pollini, Merine (Le) (IT); Federica Paladini, Surbo (Le) (IT); Gianluca Accorsi, Nardo (Le) (IT); Alessandro Sannino, Lecce (IT); Giuseppe Gigli, Lecce (IT)

(21) Appl. No.: 15/008,658

(22) Filed: Jan. 28, 2016

Related U.S. Application Data
(63) Continuation of application No. PCT/IB2014/001895, filed on Jul. 31, 2014.
(60) Provisional application No. 61/860,455, filed on Jul. 31, 2013.

(51) Int. Cl. A61K 33/38; B22F 1/00; A61K 9/51

(52) U.S. Cl. CPC A61K 33/38; A61K 9/51; B22F 1/0003; B22F 2301/255; B22F 2304/054

(57) ABSTRACT

The present invention provides methods for inhibiting or preventing cancer cell growth using silver nanoparticles.

### RELATED APPLICATION

This application is a continuation application of International Application No. PCT/IB2014/001895, which designated the United States and was filed on Jul. 31, 2014, published in English, which claims the benefit of U.S. Provisional Application No. 61/860,455 filed on Jul. 31, 2013. The entire teachings of the above applications are incorporated herein by reference.

### FIELD OF THE INVENTION

The invention encompasses methods for the use of silver nanoparticles in the treatment of cancer.

### BACKGROUND OF THE INVENTION

Since the nineteenth century, silver has been employed in a variety of areas of medical research [Jo Y K, Kim B H, Jung G., Plant Dis. 2009; 93:1037-1043]. In 1884, in Germany, Carl Siegmund Franz Credé introduced the prevention of ocular infection by administering silver nitrate solution to the eyes of neonates [Dunn K, Edwards-Jones V., Burns. 2004; 30:S1-S9]. In the 1920s, colloidal silver was accepted by the US Food and Drug Administration (FDA) as being effective for wound management [Chopra I., J. Antimicrob. Chemother. 2007; 59:587-590], and through the first half of the twentieth century silver was used in controlling infection in burn wounds [Dunn K, Edwards-Jones V., Burns. 2004; 30:S1-S9].

In the 1940s, penicillin was introduced as a healing method, so antibiotics became the standard treatment for bacterial infections and the use of silver diminished [Chopra I., J. Antimicrob. Chemother. 2007; 59:587-590; Kim J, Kwon S, Ostler E., J. Biol. Eng. 2009; 3:20]. However, the resistance of pathogenic bacteria to many antibiotics and the growing interest in nanotechnologies and nano-sized materials have led to many technological advances of nano-sized silver and to the development of many applications, such as coatings for medical devices, silver dressings, silver coatings on textile fabrics [Chopra I. J. Antimicrob. Chemother. 2007; 59:587-590; Rai M, Yadav A, Gade A. Biotechnol. Adv. 2009; 27:76-83], water sanitization [Jain P, Pradeep T., Biotechnol. Bioeng. 2005; 90:59-63] etc. Colloidal silver nanoparticles have also been used as an antimicrobial and disinfectant agent. Today, even NASA uses silver to purify drink water in space flights [Dunn K, Edwards-Jones V., Burns. 2004; 30:S1-S9].

Cancer is an important cause of mortality worldwide and the number of people who are affected is increasing. Chemotherapeutic drugs are routinely used in the treatment of cancer. However, this therapy has its own critical flaws due to two major issues, namely, dose-dependent adverse conditions and the emergence of chemoresistance within the tumour. The issue of dose-dependent cumulative adverse effects derives from the pharmacological properties of cytotoxic chemotherapeutic agents, which are not tissue-specific and thus affect all tissues in a widespread manner. The emergence of chemoresistance within tumour cells is one of the main reasons for treatment failure and relapse in patients suffering from metastatic cancer conditions. Resistance of the tumour cell to chemotherapeutic agent exposure may be innate, whereby the genetic characteristics of the tumour cells are naturally resistant to chemotherapeutic drug exposure. Alternatively, chemoresistance can be acquired through development of a drug resistant phenotype over a defined time period of exposure of the tumour cell to individual/multiple chemotherapy combinations. The biological routes by which the tumour cell is able to escape death by chemotherapy are numerous and complex. Radiation therapy for cancer also has deleterious effects on the patient.

In an attempt to achieve less toxic methods of cancer treatment, and to overcome the inherent insensitivity of cancer cells to current therapies, novel therapeutic strategies are still required. Accordingly, there is a need in the art for improved methods for cancer therapy. The present invention fulfills these needs and further provides other related advantages.

### SUMMARY OF THE INVENTION

The present invention relates to methods and pharmaceutical compositions useful in the treatment of cancer.

In one embodiment, the invention provides a method for inhibiting the growth or proliferation of a cancer cell. The method comprises the step of contacting a cancer cell with a silver nanoparticle.

In another embodiment, the invention provides a method for treating a cancer in a subject in need thereof. The method comprises the step of administering to the subject a therapeutically effective amount of silver nanoparticles (“AgNps”).

In another embodiment, the invention provides the use of silver nanoparticles in the manufacture of a medicament for treating cancer in a subject in need thereof.

Additional embodiments of the invention include pharmaceutical compositions comprising silver nanoparticles which are suitable for treating cancer in a subject in need thereof.

### DESCRIPTION OF THE DRAWINGS

FIGS. 1A-1D present graphs illustrating the results of the MTT cytoviability assay (1-30 days) for human neuroblastoma cells (IMR32) interacting with culture medium (i.e., not treated, NT) and different concentration (1.5-15 ppm) of AgNps with a nominal size of 3 nm (1A), 10 nm (1B), 60 nm (1C), 100 nm (1D); representative measurements of three distinct sets of data are shown (t-Student test, P<0.05).

FIGS. 2A-2D present graphs illustrating the results of the MTT cytoviability assay (1-30 days) for human breast cancer cells (MCF7) interacting with culture medium (i.e., not treated, NT) and different concentrations (1.5-15 ppm) of AgNps with a nominal size of 3 nm (2A), 10 nm (2B), 60 nm (2C),100 nm (2D); representative measurements of three distinct sets of data are shown (t-Student test, P<0.05).

FIGS. 3A-3D present graphs illustrating the results of the MTT cytoviability assay (1-30 days) for human chronic myeloid leukemic cells (KU812) interacting with culture medium (i.e., not treated, NT) and different concentration (1.5-15 ppm) of AgNps with nominal size of 3 nm (3A), 10 nm (3B), 60 nm (3C),100 nm (3D); representative measurements of three distinct sets of data are shown (t-Student test, P<0.05).

FIGS. 4A-4D present graphs illustrating the results of the MTT cytoviability assay (1-30 days) for human fibroblasts (BJ) interacting with culture medium (i.e., not treated, NT) and different concentration (1.5-15 ppm) of AgNps with nominal size of 3 nm (4A), 10 nm (4B), 60 nm (4C), 100 nm (4D); representative measurements of three distinct sets of data are shown (tStudent test, P<0.05).

FIGS. 5A-5D present graphs illustrating the results of the MTT cytoviability assay (1-30 days) for human mammary gland cells (MCF10A) interacting with culture medium (i.e., not treated, NT) and different concentration (1.5-15 ppm) of AgNps with nominal size of 3 nm (5A), 10 nm (5B), 60 nm (5C), 100 nm (5D); representative measurements of three distinct sets of data 5 are shown (t-Student test, P<0.05).

FIGS. 6A-6D present graphs illustrating the results of the MTT cytoviability assay (1-30 days) for human B lymphoblast cells (C13589) interacting with culture medium (i.e., not treated, NT) and different concentration (1.5-15 ppm) of AgNps with nominal size of 3 nm (6A), 10 nm (6B), 60 nm (6C), 100 nm (6D); representative measurements of three distinct sets of data 10 are shown (t-Student test, P<0.05).

FIG. 7 illustrates an MTT cell viability assay for human chronic myeloid leukemia cells (KU812) using different concentration of silver nanoparticles (AgNps) and a media control (not treated, NT). Samples were treated for 24 hours with various concentrations of silver nanoparticles (AgNps), ranging from 0.25 ppm to 15 ppm.

FIG. 8 is a graph showing the inhibition rate (%) of superoxide dismutase activity in AgNps (3, 10, 60, 100 nm) treated KU812 and C13589 cells for 6 hours. The experiments were performed in triplicate; data shown represent mean±SD of three independent experiments (t-Student test, P<0.05 as compared with untreated cells, NT).

FIG. 9 is a graph showing nitric oxide production in AgNps (3, 10, 60, 100 nm) treated KU812 and C13589 cells for 6 hours. The experiments were performed in triplicates; data shown represent mean±SD of three independent experiments (t-Student test, P<0.05 as compared with untreated cells, NT).

FIGS. 10A-10H present fluorescent images of intracellular uptake of AgNps 3, 10, 60, 100 nm coated with PAH-TRICT by (10A,10B,10C,10D) human chronic myeloid leukemia cells (KU812) and (10E,10F,10 G,10H) normal human B lymphocyte cells (C13589).

FIGS. 11A-11I present TEM images of ultrathin sections of KU812 cells treated with AgNps with size 3 nm (1.5 ppm).

FIG. 12A is an agarose electrophoresis gel of DNA isolated from AgNps treated KU812 leukemia cells.

FIG. 12B is an agarose electrophoresis gel of DNA isolated from AgNps treated healthy C13895 cells.

### DETAILED DESCRIPTION OF THE INVENTION

In one embodiment, the invention relates to a method for inhibiting the growth or proliferation of cancer cells, comprising contacting the cancer cells with an effective amount of silver nanoparticles. Preferably, the cancer cells are in the body of a subject.

In another embodiment, the invention relates to a method for treating cancer in a subject in need thereof. The method comprises the step of administering to the subject an effective amount of silver nanoparticles.

Preferably, the silver nanoparticles of the invention have anti-cancer effects without having deleterious effects on normal cells.

As used herein, the term “cancer cells” is equivalent to the term “tumor cells”. Cancer cells can be in the form of a tumor, exist alone within a subject (e.g., leukemia cells), or can be cell lines derived from a cancer.

As used herein, a “therapeutically effective amount” of silver nanoparticles is an amount which is effective for treating, alleviating, ameliorating, relieving, delaying onset of, inhibiting progression of, reducing severity of, and/or reducing incidence of one or more symptoms or features of cancer. In preferred embodiments, a therapeutically effective amount is effective to prevent or reduce cancer symptoms, reduce tumor size, prevent or reduce metastasis, prevent or reduce tumor growth, eliminate the presence of the tumor or cancer cells, render a cancer cell unviable, or is cytotoxic to the tumor cells.

In preferred embodiments, the silver nanoparticles are incorporated into a vehicle suitable for administration to a subject and/or for delivery to a cancer cell.

In some embodiments, the silver nanoparticles of the present invention inhibit the growth of cancer cells. As used herein, the term “inhibits growth of cancer cells” or “inhibiting growth of cancer cells” refers to any slowing of the rate of cancer cell proliferation and/or migration, arrest of cancer cell proliferation and/or migration, killing of cancer cells, or reducing cell viability, such that the rate of cancer cell growth is reduced in comparison with the observed or predicted rate of growth of an untreated control cancer cell. The term “inhibits growth” can also refer to a reduction in size or disappearance of a cancer cell or tumor, as well as to a reduction in its metastatic potential. Preferably, such an inhibition at the cellular level may reduce the size, deter the growth, reduce the aggressiveness, or prevent or inhibit metastasis of a cancer in a patient. Those skilled in the art can readily determine, by any of a variety of suitable indicia, whether cancer cell growth is inhibited.

Inhibition of cancer cell growth may be evidenced, for example, by arrest of cancer cells in a particular phase of the cell cycle, e.g., arrest at the G2/M phase of the cell cycle, or by measuring the decrease in mitochondrial activity using an MTT [(3-(4,5-dimethylthiazol-2-yl)-2,5-diphenyltetrazolium bromide)] assay. Inhibition of cancer cell growth can also be evidenced by direct or indirect measurement of cancer cell or tumor size. In human cancer patients, such measurements generally are made using well known imaging methods such as magnetic resonance imaging, computerized axial tomography and X-rays. Cancer cell growth can also be determined indirectly, such as by determining the levels of circulating carcinoembryonic antigen, prostate specific antigen or other cancer-specific antigens that are correlated with cancer cell growth. Inhibition of cancer growth is also generally correlated with prolonged survival and/or increased health and well-being of the subject.

In some embodiments, the method of treating cancer of the invention comprises administering to the subject a therapeutically effective amount of silver nanoparticles in such amounts and for such time as is necessary to achieve the desired result.

As used herein, the term “nanoparticle” refers to a nanostructure that is typically between about 0.1 nm and 400 nm across the largest dimension of the structure. A nanoparticle of the invention may be spherical, oblong, tubular, cylindrical, cubic, hexagonal, dumbbell or any other shape that may be envisaged or built in a laboratory setting. A silver nanoparticle of the invention is typically from about 0.1 nm to about 400 nm in its largest dimension, but in some instances, may be bigger or smaller. In another embodiment, the average size of a plurality of silver nanoparticles in a composition is from about 0.1 nm and 400 nm across the largest dimension. In a preferred embodiment the largest dimension of the silver nanoparticles is from about 1 nm to about 100 nm. In one embodiment, in compositions comprising a multiplicity of silver nanoparticles, the largest dimensions of the nanoparticles have a size distribution centered at about 1 nm to about 100 nm.

The silver nanoparticles preferably do not include any targeting or therapeutic agent attached thereto.

In some embodiments, the method comprises administering to the subject a composition comprising silver nanoparticles at a concentration of between about 0.1 parts per million (ppm) and 15 ppm by weight. In a preferred embodiment, the silver nanoparticles are at a concentration of between about 1 ppm and 25 ppm. In one embodiment, the silver nanoparticles are present in an aqueous suspension, such as a colloidal suspension, that further comprises a stabilizer. Examples of stabilizers include, but are not limited to, propylene glycol and aqueous sodium citrate. In a preferred embodiment, the stabilizer is at least about 0.5% propylene glycol or sodium citrate by weight.

In some embodiments, the cell contacted in the method of the invention is an in vitro cell line. In some alternative embodiments, the cell line may be a primary cell line. Methods of preparing a primary cell line utilize standard techniques known to individuals skilled in the art. In other alternatives, a cell line may be an established cell line. A cell line may be adherent or non-adherent, or a cell line may be grown under conditions that encourage adherent, non-adherent or organotypic growth using standard techniques known to individuals skilled in the art. A cell line may be contact inhibited or non-contact inhibited. In exemplary embodiments, a cell line is an established human cell line derived from a tumor. Non-limiting examples of cell lines derived from a tumor may include the osteosarcoma cell lines 143B, CAL-72, G-292, HOS, KHOS, MG-63, Saos-2, and U-20S; the prostate cancer cell lines DU145, PC3 and Lncap; the breast cancer cell lines MCF-7, MDA-MB-438 and T47D; the myeloid leukemia cell lines KU812 and THP-1, the glioblastoma cell line U87; the neuroblastoma cell lines IMR32 and SHSY5Y; the bone cancer cell line Saos-2; and the pancreatic carcinoma cell line Panc1. In exemplary embodiments, cells contacted by the method of the invention are derived from the human neuroblastoma cell line IMR32, the human breast cancer cell line MCF7, and the human chronic myeloid leukemia cell line KU812. Methods of culturing cell lines are known in the art.

In other embodiments, the cell is contacted by the method of the invention in vivo. Suitable subjects include, but are not limited to, mammals, amphibians, reptiles, birds, fish, and insects. In preferred embodiments, the subject is a human.

The silver nanoparticles can be administered to the subject in a variety of ways, such as parenterally, intraperitoneally, intravascularly, intratumorally or intrapulmonarily, preferably in dosage unit formulations containing one or more nontoxic pharmaceutically acceptable carriers, adjuvants, and vehicles as desired. The term “parenteral” as used herein includes subcutaneous, intravenous, intramuscular, intrathecal, or intrasternal injection, or infusion techniques. As used herein, the term “pharmaceutically acceptable carrier” means a non-toxic, inert solid, semi-solid or liquid filler, diluent, encapsulating material or formulation auxiliary of any type. Remington's Pharmaceutical Sciences. Ed. by Gennaro, Mack Publishing, Easton, Pa., 1995 discloses various carriers used in formulating pharmaceutical compositions and known techniques for the preparation thereof. Some examples of materials which can serve as pharmaceutically acceptable carriers include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as TWEEN™ 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. If filtration or other terminal sterilization methods are not feasible, the formulations can be manufactured under aseptic conditions.

Injectable preparations, for example, sterile injectable aqueous or oleaginous suspensions, may be formulated according to the known art using suitable dispersing or wetting agents and suspending agents. The sterile injectable preparation may also be a sterile injectable solution or suspension in a nontoxic parenterally acceptable diluent or solvent. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid are useful in the preparation of injectables. Dimethyl acetamide, surfactants including ionic and non-ionic detergents, and polyethylene glycols can be used. Mixtures of solvents and wetting agents such as those discussed above are also useful.

The method of the invention may be used to treat a neoplasm or a cancer. The term “cancer” includes pre-malignant as well as malignant cancers. The neoplasm or cancer can be malignant or benign. The cancer can be primary or metastatic; the neoplasm or cancer may be early stage or late stage. Non-limiting examples of neoplasms or cancers that can be treated by the methods and compositions of the invention include, but are not limited to, acute lymphoblastic leukemia, acute myeloid leukemia, adrenocortical carcinoma, AIDS-related cancers, AIDS-related lymphoma, anal cancer, appendix cancer, astrocytomas (childhood cerebellar or cerebral), basal cell carcinoma, bile duct cancer, bladder cancer, bone cancer, brainstem glioma, brain tumors (cerebellar astrocytoma, cerebral astrocytoma/malignant glioma, ependymoma, medulloblastoma, supratentorial primitive neuroectodermal tumors, visual pathway and hypothalamic gliomas), breast cancer, bronchial adenomas/carcinoids, Burkitt lymphoma, carcinoid tumors (childhood, gastrointestinal), carcinoma of unknown primary, central nervous system lymphoma (primary), cerebellar astrocytoma, cerebral astrocytoma/malignant glioma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma in the Ewing family of tumors, extracranial germ cell tumor (childhood), extragonadal germ cell tumor, extrahepatic bile duct cancer, eye cancers (intraocular melanoma, retinoblastoma), gallbladder cancer, gastric (stomach) cancer, gastrointestinal carcinoid tumor, gastrointestinal stromal tumor, germ cell tumors (childhood extracranial, extragonadal, ovarian), gestational trophoblastic tumor, gliomas (adult, childhood brain stem, childhood cerebral astrocytoma, childhood visual pathway and hypothalamic), gastric carcinoid, hairy cell leukemia, head and neck cancer, hepatocellular (liver) cancer, Hodgkin lymphoma, hypopharyngeal cancer, hypothalamic and visual pathway glioma (childhood), intraocular melanoma, islet cell carcinoma, Kaposi sarcoma, kidney cancer (renal cell cancer), laryngeal cancer, leukemias (acute lymphoblastic, acute myeloid, chronic lymphocytic, chronic myelogenous, hairy cell), lip and oral cavity cancer, liver cancer (primary), lung cancers (non-small cell, small cell), lymphomas (AIDS-related, Burkitt, cutaneous T-cell, Hodgkin, non-Hodgkin, primary central nervous system), macroglobulinemia (Waldenstrom), malignant fibrous histiocytoma of bone/osteosarcoma, medulloblastoma (childhood), melanoma, intraocular melanoma, Merkel cell carcinoma, mesotheliomas (adult malignant, childhood), metastatic squamous neck cancer with occult primary, mouth cancer, multiple endocrine neoplasia syndrome (childhood), multiple myeloma/plasma cell neoplasm, mycosis fungoides, myelodysplastic syndromes, myelodysplastic/myeloproliferative diseases, myelogenous leukemia (chronic), myeloid leukemias (adult acute, childhood acute), multiple myeloma, myeloproliferative disorders (chronic), nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer (surface epithelial-stromal tumor), ovarian germ cell tumor, ovarian low malignant potential tumor, pancreatic cancer, pancreatic cancer (islet cell), paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors (childhood), pituitary adenoma, plasma cell neoplasia, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma (childhood), salivary gland cancer, sarcoma (Ewing family of tumors, Kaposi, soft tissue, uterine), Sezary syndrome, skin cancers (nonmelanoma, melanoma), skin carcinoma (Merkel cell), small cell lung cancer, small intestine cancer, soft tissue sarcoma, squamous cell carcinoma, squamous neck cancer with occult primary (metastatic), stomach cancer, supratentorial primitive neuroectodermal tumor (childhood), T-Cell lymphoma (cutaneous), testicular cancer, throat cancer, thymoma (childhood), thymoma and thymic carcinoma, thyroid cancer, thyroid cancer (childhood), transitional cell cancer of the renal pelvis and ureter, trophoblastic tumor (gestational), enknown primary site (adult, childhood), ureter and renal pelvis transitional cell cancer, urethral cancer, uterine cancer (endometrial), uterine sarcoma, vaginal cancer, visual pathway and hypothalamic glioma (childhood), vulvar cancer, Waldenstrom macroglobulinemia, and Wilms tumor (childhood).

The silver nanoparticles can be administered to the subject in combination with one or more additional anti-cancer therapies, such as radiation or a chemotherapeutic agent.

In some embodiments, the composition of the invention comprises a vehicle for cellular delivery. In these embodiments, the silver nanoparticles are encapsulated in a suitable vehicle to either aid in the delivery of the nanoparticles to target cells, to increase the stability of the nanoparticles, or to minimize potential toxicity of the nanoparticles. A variety of vehicles are suitable for delivering the silver nanoparticles. Non-limiting examples of suitable structured fluid delivery systems include polyethylene glycol, liposomes, microemulsions, micelles, dendrimers and other phospholipid-containing systems. Liposomes may further comprise a suitable solvent. The solvent can be an organic solvent or an inorganic solvent. Suitable solvents include, but are not limited to, dimethylsulfoxide (DMSO), methylpyrrolidone, N-methylpyrrolidone, acetronitrile, alcohols, dimethylformamide, tetrahydrofuran, or combinations thereof. Methods of incorporating compositions into delivery vehicles are known in the art.

The silver nanoparticles of the invention can be formulated in unit dosage form for ease of administration and uniformity of dosage. The expression “unit dosage form”, as used herein, refers to a physically discrete amount or mass of nanoparticles appropriate for treatment of the subject. The dosing of the silver nanoparticle compositions will be determined by the attending physician within the scope of sound medical judgment.

The therapeutically effective dose can be estimated initially using methods known the art, for example in cell culture assays or in animal models, for example in mice, rabbits, dogs, or pigs. Animal models can also be used to determine an effective concentration range and route of administration. Such information can then be used to determine useful doses and routes for administration in humans. Therapeutic efficacy and toxicity of silver nanoparticles can be determined by standard pharmaceutical procedures in cell cultures or experimental animals, e.g., ED₅₀ (the dose is therapeutically effective in 50% of the population) and LD₅₀ (the dose is lethal to 50% of the population). The dose ratio of toxic to therapeutic effects is the therapeutic index, and it can be expressed as the ratio, LD₅₀/ED₅₀. The data obtained from cell culture assays and animal studies can be used in formulating a range of dosage for human use.

The following examples are intended to illustrate certain embodiments of the present invention, but do not exemplify the full scope of the invention.

### EXAMPLES

### Example 1

### Characterization of Silver Nanoparticles

AgNps with a nominal size of 3 nm (TEM charcterization) were obtained from ClusterNanoTech Ltd in aqueous buffer and stabilized in a 0.5% propylene glycol solution. AgNps with nominal sizes of 10, 60 and 100 nm (TEM characterization) were obtained from Sigma-Aldrich in aqueous buffer and stabilized in sodium citrate.

The AgNps were subjected to an extensive characterization process, with measurements performed on AgNps as purchased and on test suspensions of AgNps. The suspensions of AgNps were prepared in water (Millipore, 18.2 MΩ cm) and culture medium at 25° C. using a bath-sonicator prior to size and zeta potential measurements. Dynamic light scattering (DLS) and zeta-potential (ζ) measurements were performed on a Zetasizer Nano ZS90 (Malvern, Pa., USA) equipped with a 4.0 mW He-Ne laser operating at 633 nm and an avalanche photodiode detector.

Table 1 shows the number size average of 20 ppm AgNps in water and culture medium. For the DLS measurements in culture medium, the AgNps were incubated for 24 hours in culture medium at 37° C. The increase in apparent size in culture medium can be attributed to changes in the hydrodynamic radius of the particle in the culture medium due to particle and medium components interaction.

TABLE 1
Table 1. Size measurement of 20 ppm of AgNps in
water and culture medium. Data shown represent
mean ± SD of three independent measurements.

| Nominal size | Water | Culture medium |
|---|---|---|
| 3 nm | 3.49 nm ± 1.12 | 36.56 nm ± 0.021 |
| 10 nm | 9.86 nm ± 0.02 | 50.92 nm ± 0.47 |
| 60 nm | 56.4nm ± 3.80 | 107.55 nm ± 1.90 |
| 100 nm | 89.41 nm ± 0.85 | 148.85 nm ± 2.75 |

The average of zeta potential AgNps at 20 ppm in water and in culture medium is shown in Table 2.

TABLE 2
Table 2. Zeta potential measurement of 20 ppm of
AgNps in water and culture medium. Data shown represent
mean ± SD of three independent measurements.

| AgNps | Water | Culture medium |
|---|---|---|
| 3 nm | −0.85 mV ± 0.17 | −9.09 mV ± 0.78 |
| 10 nm | −1.45 mV ± 0.78 | −2.93 mV ± 0.31 |
| 60 nm | −1.30 mV ± 0.98 | −6.68 mV ± 0.31 |
| 100 nm | −0.34 mV ± 0.12 | −10.99 mV ± 2.04 |

The physiochemical characteristics of nanoparticles play a significant role in their effects on biological systems. The principal parameters of nanoparticles are their shape, size, and the morphological sub-structure of the substance. The zeta potential of the particle has been reported to play a significant role in its interaction with different biomolecules (Vila, A., Sanchez, A., Tobío, M., Calvo, P., Alonso, M. J., 2002. J. Control. Release 78, 15-24) and the change in the zeta potential in the exposure medium has been shown to correlate well with toxic response (Mukherjee, S. P., Davoren, M., Byrne, H. J., 2010, Toxicol. In Vitro 24 (1), 1169-1177). The size measurement of AgNps by DLS technique shows increased diameter after dispersal in the cell culture medium supplemented with 10% FBS. This indicates possible interaction of AgNps with components of the cell culture medium, which have been widely reported with different nanoparticles to lead to the formation of ‘protein corona’ (Lynch, I., Dawson, K., 2008, Nanotoday 3, 40-47; Lundqvist, M., Stigler, J., Elia, G., Lynch, I., Cedervall, T., Dawson, K., 2008, PNAS 105, 14265-14270).

The zeta potential study also shows a decrease in the negative zeta potential of the AgNPs upon dispersal in the 10% FBS supplemented cell culture media. Interaction of single walled carbon nanotubes with the components of cell culture medium has been shown to elicit a secondary or indirect toxic response (Casey, A., Davoren, M., Herzog, E., Lyng, F. M., Byrne, H. J., Chambers, G., 2007, Carbon 45, 34-40; Casey, A., Herzog, E., Lyng, F. M., Byrne, H. J., Chambers, G., Davoren, M., 2008, Toxicol. Lett. 179, 78-84) and there may be similar contributions to the toxic response observed here.

### Example 2

### Silver Nanoparticles are Cytotoxic to Bacterial Cells

To verify the effective cytotoxic potential of silver nanoparticles, an antibacterial assay was performed. In order to quantify the bacterial reduction induced by the different amounts of silver nanoparticles (1.5, 6 and 15 ppm), bacterial counts on Escherichia coli (DH5(α), inoculating cell density 9.1*10⁶ CFU/ml were performed through serial dilution methods. Samples were incubated in 4 ml of Luria Broth inoculated with 100 microliters of bacterial suspension for 24 hours at 37° C. in triplicate. After incubation, serial dilutions were performed in 0.85% sterile saline. One hundred microliters of each dilution was plated in duplicate on agar plates and the dishes were incubated for 24 hours at 37° C. The results were expressed as percentage of bacteria reduction rate. The results obtained were 57%, 60%, and 63% for samples with a concentration of 1.5, 6 and 15 ppm of silver nanoparticles, respectively.

### Example 3

### Silver Nanoparticles are Cytotoxic to Human Cancer Cells, but Not to Normal Human Cells, In Vitro

Viability assays can explain the cellular response to a toxicant. They also give information on cell death, survival, and metabolic activities. The toxicity of AgNps was assessed by the decrease in mitochondrial activity using the MTT assay in different human normal and cancer cell lines. In particular, normal or cancer cells (10⁵ cells/ml) were incubated at 37° C. in 5% CO₂, 95% relative humidity for 1,2,3,8 and 30 days with a colloidal AgNps (0.25-15 ppm) suspension. The control was complete culture medium only. After an appropriate incubation period, cultures were removed from the incubator and MTT solution was added in an amount equal to 10% of the culture volume. The cultures were returned to the incubator and incubated for 3 hours. After the incubation period, the cultures were removed from the incubator and the resulting MTT formazan crystals were dissolved in a volume of acidified isopropanol solution equal to the culture volume. The plates were read within 1 hour after adding acidified isopropanol solution. Spectrophotometrically measure absorbance a wavelength of 570 nm. Background absorbance measured at 690 nm was subtracted. The percentage viability was expressed as the relative growth rate (RGR) by the equation:

RGR=(Dₛₐₘₚₗₑ/D_{control})*100%

where Dₛₐₘₚₗₑ and D_{control} are the absorbances of the sample and the negative control. Each assay was performed in triplicate.

It was important to assess cytotoxicity of the AgNps upon 24 hours of incubation since the cells would be in an exponential growth phase during this period and any toxicity that reflects inhibition of proliferation and/or cell death would be clearly visible (N. Nafee, M. Schneider, U. F. Schaefer, and C. M. Lehr, International Journal of Pharmaceutics, vol. 381, no. 2, pp. 130-139, 2009).

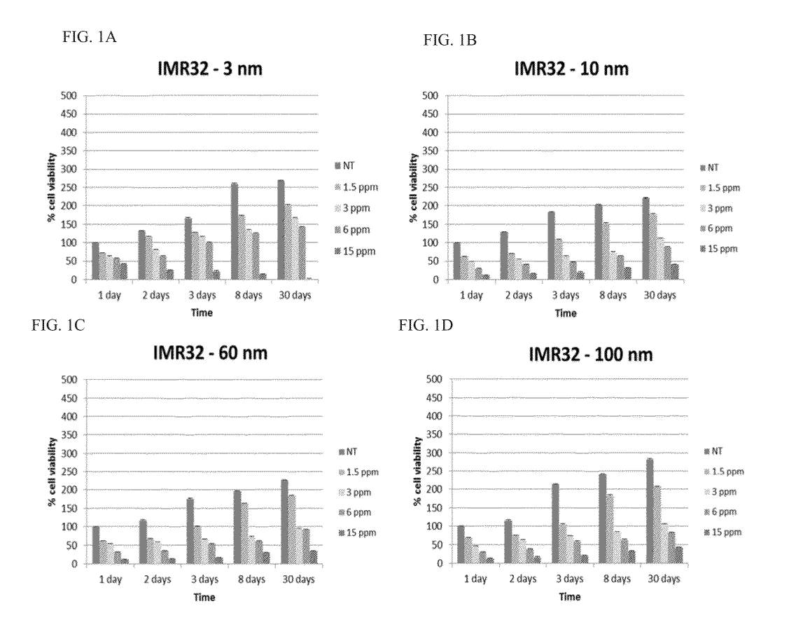

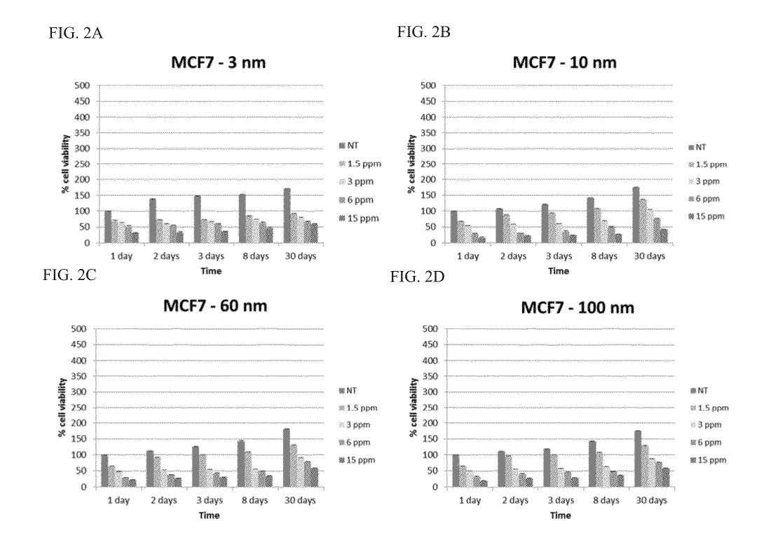

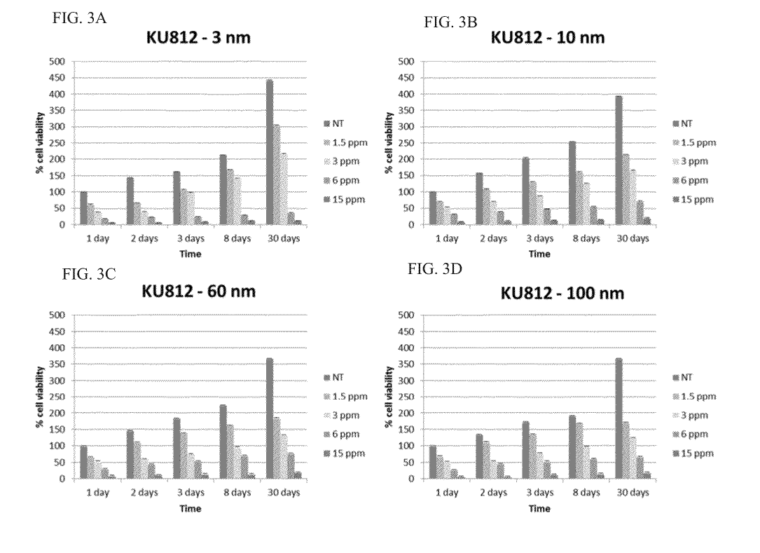

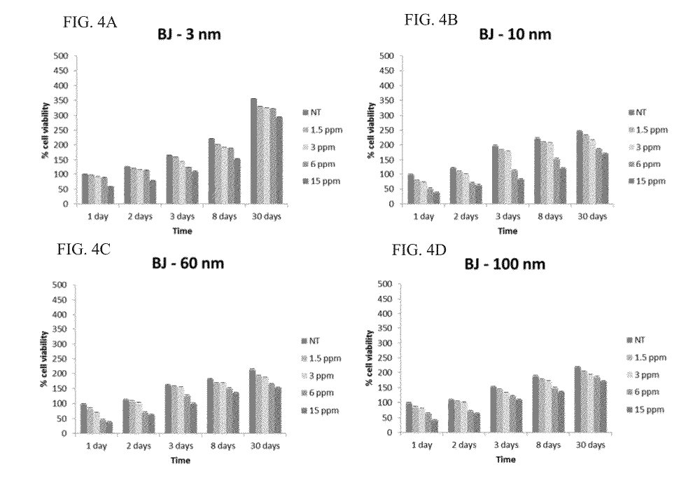

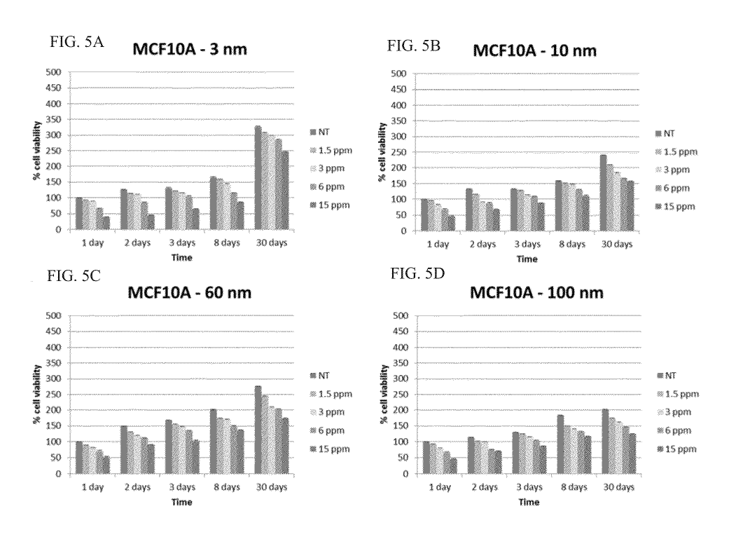

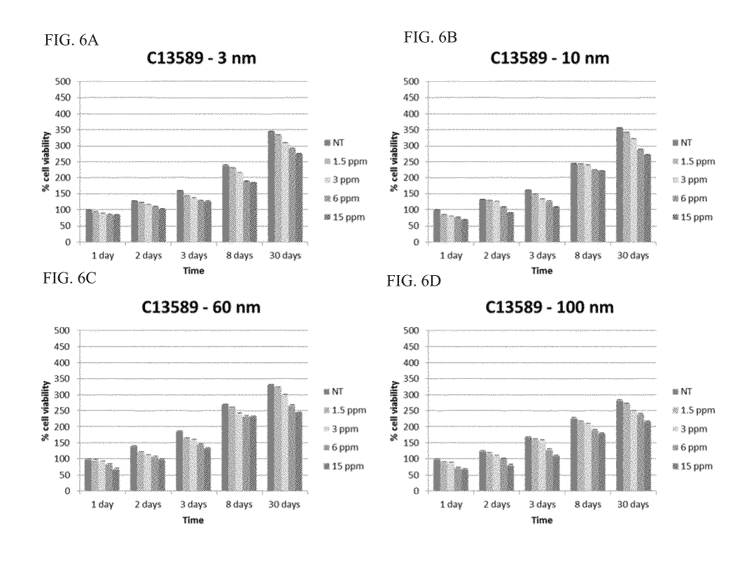

The MTT assay determines the ability of viable cell's mitochondria to reduce the soluble, yellow MTT into insoluble, purple formazan. The reduction of MTT to formazan indicates the decrease in mitochondrial metabolism of the cells. Therefore, the absorbance of formazan formed directly correlates to the number of cells whose mitochondrial metabolism is intact even after exposure to AgNps. A reduction in mitochondrial function of cancer cells exposed to AgNps for 1-30 days was observed in a dose dependent manner (1.5-15 ppm). Our in vitro studies showed that colloidal silver induced a dose-dependent cell death in different cancer cell lines, as human neuroblastoma, IMR32 (FIGS. 1A-1D), human breast cancer, MCF7 (FIGS. 2A-2D) and human chronic myeloid leukemia cells, KU812 (FIGS. 3A-3D), without affecting the viability of normal control cells, as human fibroblast, BJ (FIGS. 4A-4D), human mammary gland, MCF10A (FIGS. 5A-5D) and human B lymphoblast, C13589 (FIGS. 6A-6D). In particular, the size of AgNps did not affect their cytotoxicity toward cancer cells.

### Example 4

### Median Lethal Dose (LD50) of AgNps on Human Chronic Myeloid Leukemia Cells (KU812)

The median lethal dose (LD₅₀) and lethal dose (LD₁₀₀) of AgNps on human chronic myeloid leukemia cells (KU812) was determined. Cell viability was determined by MTT assay at 24 hours to treatment with escalation dose of AgNps. Representative measurements are of three distinct data sets (Student-t test, P<0.05).

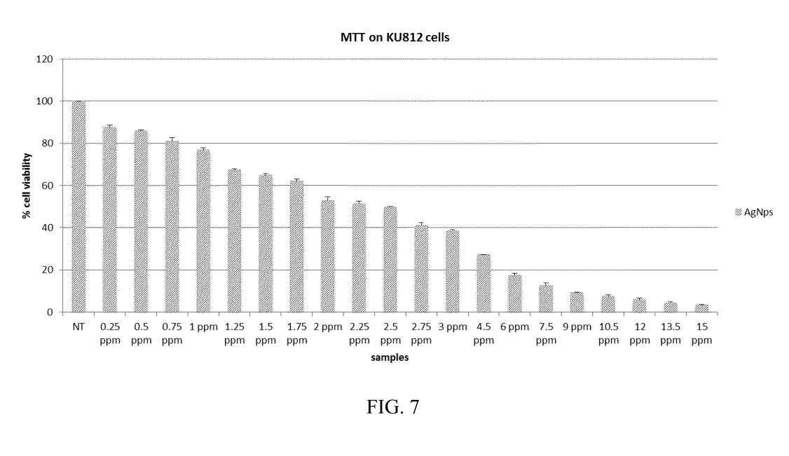

As observed in FIG. 7, silver nanoparticles induced a dose-dependent cytotoxic effect on KU812 cells, the median lethal dose (LD₅₀) was in the range between 1.5-2.5 ppm, and the lethal dose (LD₁₀₀) was in the range between 12-15 ppm. The LD₅₀ values determined were used in subsequent experiments.

### Example 5

### Silver Nanoparticle-Induced Formation of Reactive Oxygen Intermediates

Cell death can be produced by Reactive Oxygen Intermediates (ROI) and Reactive Nitrogen Intermediates (RNI) metabolites. Superoxide dismutase (SOD), which catalyzes the dismutation of the superoxide anion (O₂⁻) into hydrogen peroxide and molecular oxygen, is one of the most important antioxidative enzymes.

Antioxidant production was measured using a superoxide dismutase (SOD) assay kit (Sigma-Aldrich, USA) according to the manufacturer's instructions. Briefly, to determine the activity of SOD, human chronic leukemia cells (KU812) and normal human B lymphocyte cells (C13589) were incubated with the LD₅₀ (1.5 ppm) of AgNps (3, 10, 60, 100 nm) for 6 hours. Cells were then washed three times with PBS and sonicated on ice in a bath-type ultrasonicator (80 Watts outpower) for 15-s periods for a total of 4 min.; the solution was then centrifuged at 1500 rpm for 5 min. at 4° C. The resulting supernatants were used to determine intracellular antioxidants using a spectrophotometer at 440 nm. Each assay was performed in triplicate.

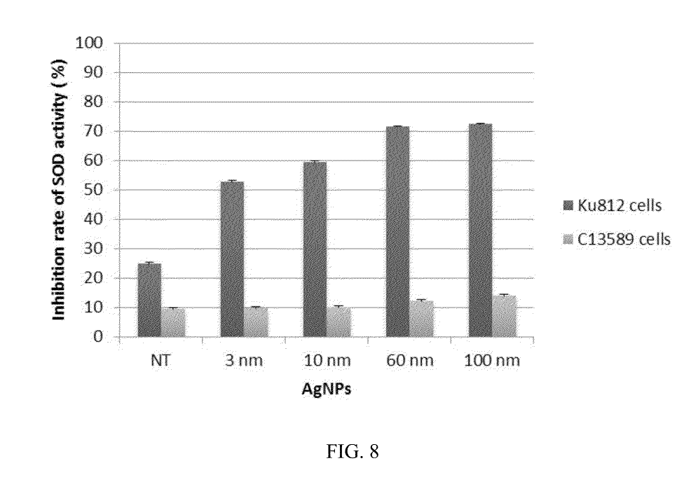

The inhibition rate of superoxide dismutase activity was significantly increased in AgNps treated KU812 cells at LD₅₀ concentrations, compared with untreated control cells (NT) and normal C13589 cell line, as show in FIG. 8.

In addition, accumulation of nitrite in the supernatants of control and treated KU812 and C13589 cells was used as an indicator of nitric oxide production. Cells were incubated for 6 hours in the presence (LD₅₀ concentration) or absence (NT) of AgNps in triplicate. After incubation, supernatants were obtained and nitrite levels were determined with the Griess reagent (Sigma-Aldrich, USA), using NaNO₂ as standard. Absorbance was spectrophotometrically measured at 540 nm wavelength.

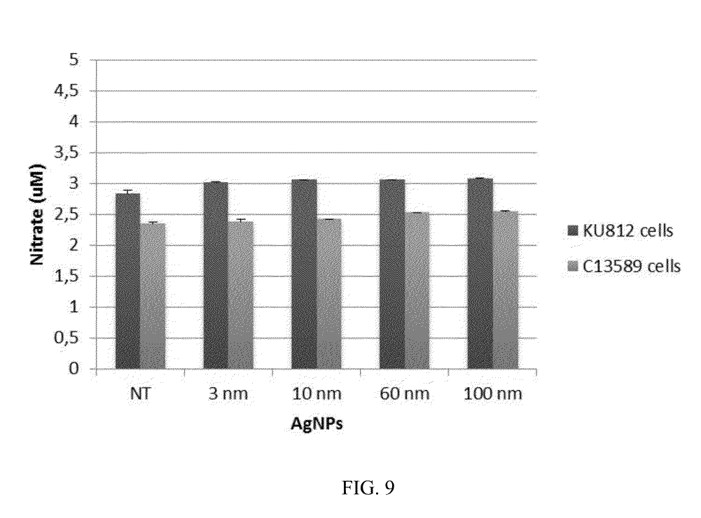

FIG. 9 shows that NO production was imperceptible in untreated C13589 cells and in AgNps treated C13589 cells at LD₅₀ concentration. However, in untreated KU812 cells, nitrite concentration was 2.83 μM, and AgNps treatment did not affect NO production.

Our results demonstrated that nitric oxide production was not affected by AgNps treatments, as compared with untreated cells, suggesting that the KU812 leukemia cell death was independent of nitric oxide production. Conversely, AgNps treatment increased the inhibition rate of superoxide dismutase activity compared with untreated KU812 and C13589 cells. This may cause a redox imbalance, significantly increasing the SOD activity in response to the production of high levels of ROI molecules and may allow the toxic effect of hydrogen peroxide (H₂O₂) leading to cell death. The H₂O₂ causes cancer cells to undergo apoptosis, pyknosis, and necrosis. In contrast, normal cells are considerably less vulnerable to H₂O₂. The reason for the increased sensitivity of cancer cells to H₂O₂ is not clear but may be due to lower antioxidant defences. In fact, a lower capacity to destroy H₂O₂ e.g., by catalase, peroxiredoxins, and GSH peroxidases may cause cancer cells to grow and proliferate more rapidly than normal cells in response to low concentrations of H₂O₂. It is well known that H₂O₂ exerts dose-dependent effects on cell function, from growth stimulation at very low concentrations to growth arrest, apoptosis, and eventually necrosis as H₂O₂ concentrations increase (Mazurek S, Zander U, Eigenbrodt E, Cell Physiol 1992, 153(3):539-49). This dose dependency may be shifted to the left in tumor cells, making them more sensitive to both the growth stimulatory and cytotoxic effects of H₂O₂. Whatever the exact mechanism, the increased sensitivity of tumor cells to killing by H₂O₂ may provide the specificity and “therapeutic window” for the antitumor therapy (Balz Frei, Stephen Lawson, PNAS 2008,105(32):11037-11038).

### Example 6

### Uptake of Silver Nanoparticles by Normal and Leukemia Cells

Uptake of AgNps by human chronic leukemic cells (KU812) and normal human B lymphocyte cells (C13589) was evaluated with fluorescent microscopy and TEM analysis. For the fluorescent microscopy analysis, the AgNps were coated with a single layer of poly-allylamine sulphate (PAH)-TRITC (1 mg/mL in NaCl 0.1 M) in order to make a fluorescent AgNps. The successful coating with PAH-TRITC were confirmed by change in zeta potential values.

Both cell lines, KU812 and C13589 cells, were seeded at a density of 1×10⁶ cells/mL and incubated with 1.5 ppm of AgNps coated with PAH-TRITC. After 24 hours of incubation at 37° C., the culture medium was removed, and the cells were washed three times with phosphate buffered saline. For fluorescent microscopic observation, cells were fixed in situ for 5 minutes in 3.7% formaldehyde and mounting with fluoroshield with DAPI (Sigma-Aldrich, USA). The samples were examined using an Olympus BX61 fluorescent microscope and imaged with a 20×, 40× and 100× objective.

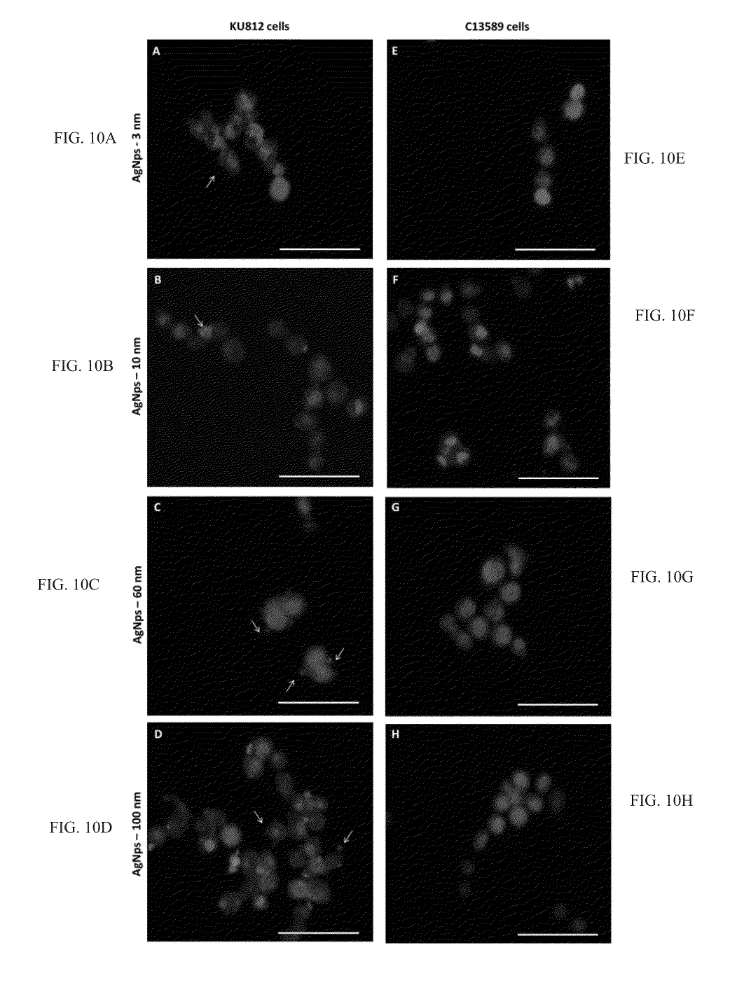

The presence of PAH-TRITC allowed the AgNps uptake and localization into cancer cells (KU812) and normal cells (C13589) to be followed after 24 hours of incubation at a concentration of 1.5 ppm. After 24 hours of incubation, strong red fluorescent staining was observed, which means AgNps have been delivered into KU812 and C13589 cells. (FIGS. 10E to 10H). The DAPI fluorescence of nuclei was shown in blue. FIGS. 10A-10D show KU812 cells following treatment with AgNps. White arrows in FIGS. 10A, 10C, and 10D indicate blebs of apoptotic KU812 cells after 24 hours of treatment with 1.5 ppm of AgNps. In FIG. 10B, the arrow indicates nuclear fragmentation.

In addition, the appearance of apoptotic bodies and characteristic cell membrane blebbing of leukemia KU812 cells due to apoptosis after treatments is also indicated by white arrows in FIGS. 10A to 10D. In contrast, the morphology of C13589 cells appeared well preserved suggesting no cellular apoptosis after incubation with same dose of AgNps (1.5 ppm), as shown in FIGS. 10E to 10H.

Ultrathin sections of the KU812 cells were analysed using tunnelling electron microscopy (TEM) to reveal the biodistribution of AgNps. Briefly, KU812 cells (2×10⁶ cells) were treated with AgNps at 1.5 ppm with size of 3 nm for 24 hours. At the end of the incubation period, cells were washed many times with phosphate buffer saline (PBS 1×) to get rid of excess unbound nanoparticles. Cells were fixed in 2.5% glutaraldehyde in 0.1 M cacodylate buffer for 30 min. Fixed cells were washed three times with cacodylate buffer. Post-fixation staining was done using 1% osmium tetroxide for 1 hour at room temperature. Cells were washed three times with cacodylate buffer and dehydrated in 25, 50, 70, 95, 100% acetone and infiltrated over night with Epon resin. Resin blocks were hardened at 60° C. for 48 hours. Ultrathin sections (70 nm) were cut using PT-PC PowerTome Ultramicrotomes (RMC products by Boeckeler, USA). The sections were stained with 1% led citrate and analysed under a JEOL Jem 1011 TEM microscope (Japan).

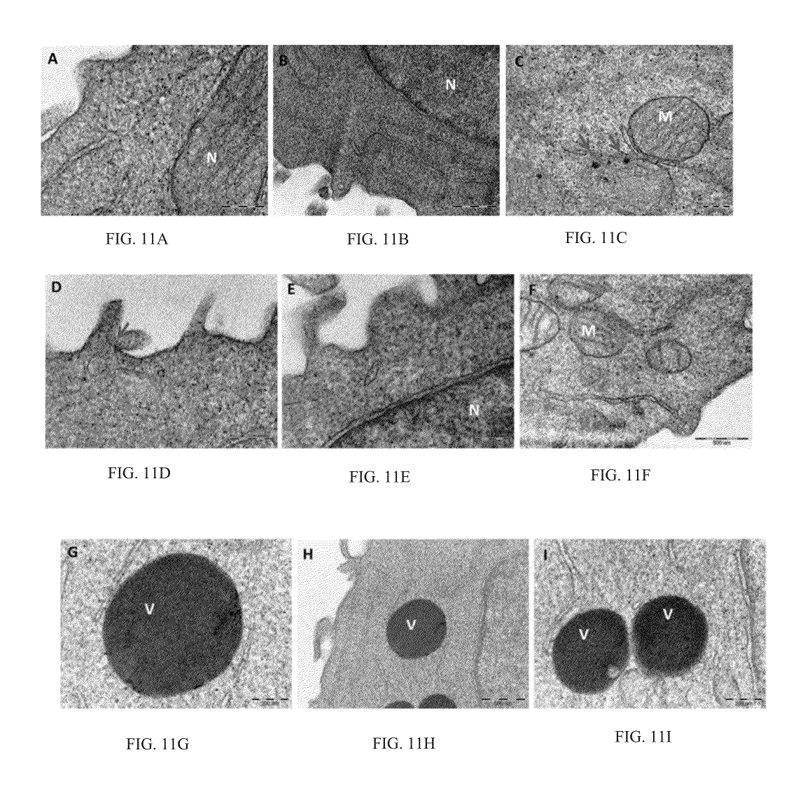

FIGS. 11A to 11I show that in AgNps treated KU812 cells, the nanoparticles were found to distributed throughout the cytoplasm (FIGS. 11A, 11C, 11D, 11E, 11F and 11G), inside mitochondria, vacuoles and nucleus. Clumps of nanoparticles found in cytoplasm were similar to nanoaggregates (red arrow in FIGS. 11C, 11D and 11G). We also observed large autophagic vacuoles with nanoparticles in the cytoplasm of the cells, as evident in FIGS. 11G, 11H and 11I. The nanoparticles were also seen deposited inside other organelles such as mitochondria (FIGS. 11C and 11F). AgNps deposition was observed in the nucleus (FIGS. 11A, 11B and 11E). This finding was in agreement with observations for other nanoparticles such as quantum dots, used as labeling and tracking tools of human leukemic cells (Garon E B, Marcu L, Luong Q, Tcherniantchouk O, Crooks G M, Koeffler H P., Leuk Res. 2007 May; 31(5):643-51.) or polyelectrolyte microcapsules (Ilaria Elena Mama, Stefano Leporatti, Emanuela De Luca, Carlo Gambacorti-Passerini, Nicola Di Renzo, Michele Maffia, Ross Rinaldi, Giuseppe Gigli, Roberto Cingolani, and Addolorata M. L. Coluccia, Nanomedicine, April 2010, Vol. 5, No. 3, 419-431) used with drug delivery systems. Owing to their small size, AgNps could be readily diffused into the nucleus through the nuclear pores. Also, the mechanism of deposition of nanoparticles in mitochondria remains unknown. The evidence of TEM images sheds light on the endocytic pathway of AgNps uptake. There are different types of active endocytosis, clathrin or caveoline mediated and macropinocytosis. The AgNps inside the cell nucleus may bind to the DNA and augment the DNA damage caused by the ROS.

Apoptosis, genetically controlled programmed cell death, has been the key criterion in the development of successful drug or gene therapy in cancer treatments. While induction of necrosis, a random event of cell lysis under extreme physiological conditions, is not favored owing to its unregulated toxic effects. In the search for newer drugs, nanoparticles are increasingly being tested for their therapeutic effects on cancer cells. Herein, we have illustrated that AgNps, with size 3-100 nm, induced apoptosis on cancerous cells to low concentration (0.25-15 ppm) any affecting the viability of healthy cells. The mitochondrial activity measurements of AgNps treated cells also imply an index of mitochondrial membrane damage during cell apoptosis.

The concentration dependent induction of AgNps mediated apoptotic pathway has immense potential application in gene therapy especially when the cells and tumors are resistant to conventional gene and drug treatments but susceptible to combined treatment with AgNps. Additionally, it is important to note that the concentration of AgNps used herein for the induction of programmed cell death is much less than the IC₅₀ values of conventional anticancer drugs. The apoptosis initiated by damage to mitochondrial membranes by AgNps is similar to the mechanism induced by other drugs or gene therapy treatments. Thus AgNps by themselves may also act as a therapeutic drug. The present findings suggest that AgNps may assume significance in the development of a suitable anticancer drug and the approach described here may lead to novel nanomedicines with strong potential in therapeutic use for treatment of cancer in conjugation with conventional drug and gene therapy.

### Example 7

### AgNps-Induced Apoptosis of Cancer Cells

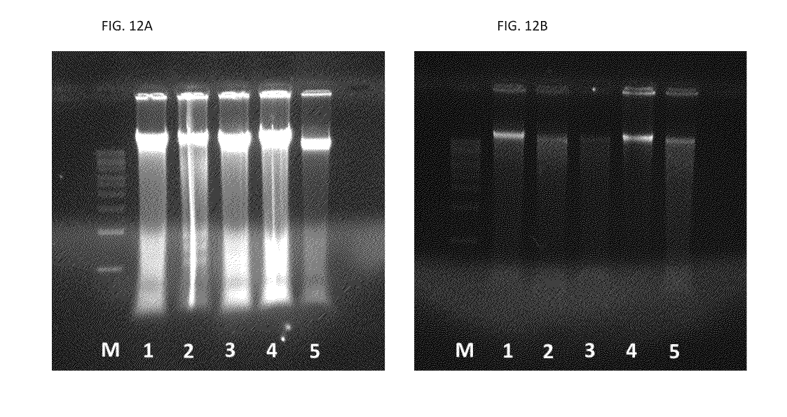

The DNA laddering technique is used to visualize the endonuclease cleavage products of apoptosis. This assay involves extraction of DNA from a lysed cell homogenate followed by agarose gel electrophoresis. Apoptosis of the AgNps treated cells was accompanied by a reduction in the percentage of cells in G0/G1 phase and an increase in the percentage of G2/M phase cells, indicating cell cycle arrest at G2/M. The ROS can act as signal molecules promoting cell cycle progression and can induce oxidative DNA damage. Further we examined the impact of AgNps in DNA fragmentation. DNA fragmentation is broadly considered as a characteristic feature of apoptosis. Induction of apoptosis can be confirmed by two factors such as irregular reduction in size of cells, in which the cells are reduced and shrunken, and lastly DNA fragmentation. The DNA fragmentation in the present study was verified by extracting DNA from C13895 healthy cells and KU812 leukemia cells treated with AgNps followed by detection in the agarose gel. FIG. 12A clearly indicates that the DNA “laddering” pattern in KU812 leukemia cells treated with AgNps is one of the reasons for cell death.

In particular, C13895 healthy cells and KU812 leukemia cells (10⁶ cells/ml) were incubated at 37° C. in 5% CO₂, 95% relative humidity for 12 hours with colloidal AgNps suspension to final concentration of 3 ppm. The control (NT) was complete culture medium only. Subsequently, the cells were lysed with lysis buffer containing 50 mM Tris HCl, pH 8.0, 10 mM ethylenediaminetetraacetic acid, 0.1 M NaCl, and 0.5% sodium dodecyl sulfate. The lysate was incubated with 0.5 mg/mL RNase A at 37° C. for one hour, and then with 0.2 mg/mL proteinase K at 50° C. overnight. Phenol extraction of this mixture was carried out, and DNA in the aqueous phase was precipitated by 1/10 volume of 7.5 M ammonium acetate and 1/1 volume isopropanol. DNA electrophoresis was performed in a 1% agarose gel containing 1 μg/mL ethidium bromide at 70 V, and the DNA fragments were visualized by exposing the gel to ultraviolet light, followed by photography.

Biochemical changes during apoptosis activate endonucleases, which cleave DNA at inter-nucleosomal linker sites to produce 180-200 bp mono- and oligo-nucleosomal fragments that gives a characteristic laddering pattern in agarose gel electrophoresis. The effects of AgNps on DNA laddering of cellular DNA fragments of KU812 leukemic cells and C13895 cells treated for 12 hours with 3 ppm of AgNps are shown in FIGS. 12A and 12B respectively. Lanes M of FIGS. 12A and 12B represent DNA marker, lanes 1 represent cells treatment with 3 nm AgNps, lanes 2 represent cells treated with 10 nm AgNps, lanes 3 represnet cells treated with 60 nm AgNps, lanes 4 represent cells treated with 100 nm AgNps and lanes 5 represent the control untreated cells (NT).

The results show the characteristic laddering pattern in AgNps treated leukemic cells (FIG. 12A) but not in healthy C13895 cells (FIG. 12B), which confirmed apoptosis as mechanism of cell death in the leukemic cells.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims. It will also be understood that none of the embodiments described herein are mutually exclusive and may be combined in various ways without departing from the scope of the invention encompassed by the appended claims.

1. A method of inhibiting the growth or proliferation of a cancer cell, comprising contacting the cancer cell with an effective amount of silver nanoparticles.

2. The method of claim 1, wherein the size of the silver nanoparticles is between about 1 nm and about 100 nm across the largest dimension.

3. The method of claim 2, wherein the silver nanoparticles are between 10 nm and 50 nm across the largest dimension.

4. The method of claim 1, wherein the silver nanoparticles are in suspension.

5. The method of claim 4, wherein the concentration of nanoparticles in suspension is from about 0.25 ppm to about 100 ppm.

6. The method of claim 1, wherein the cancer cell is selected from the group consisting of a chronic myeloid leukemia cell, a breast cancer cell, and a neuroblastoma cell.

7. A method of treating cancer in a subject in need thereof, comprising administering to the subject a therapeutically effective amount of silver nanoparticles.

8. The method of claim 7, wherein the size of the silver nanoparticles is between about 1 nm and about 100 nm across the largest dimension.

9. The method of claim 8, wherein the silver nanoparticles are between 10 nm and 50 nm 30 across the largest dimension.

10. The method of claim 7, wherein the silver nanoparticles are in suspension.

11. The method of claim 10, wherein the concentration of nanoparticles in suspension is from about 5 ppm to about 100 ppm.

12. The method of claim 7, wherein the cancer is selected from the group consisting of chronic myeloid leukemia, breast cancer and neuroblastoma.

13. A pharmaceutical composition comprising silver nanoparticles, wherein said pharmaceutical composition is suitable for parenteral administration.

14. The pharmaceutical composition of claim 13, wherein the size of the silver nanoparticles is between about 1 nm and about 100 nm across the largest dimension.

15. The pharmaceutical composition of claim 14, wherein the silver nanoparticles are between 10 nm and 50 nm across the largest dimension.

16. The pharmaceutical composition of claim 13, wherein the silver nanoparticles are in suspension.

17. The pharmaceutical composition of claim 16, wherein the concentration of nanoparticles in suspension is from about 5 ppm to about 100 ppm.
